(19) United States
(12) Patent Application Publication
(10) Pub. No.: US 2026/0283940 A1
(43) Pub. Date: Sep. 24, 2026

(54) RADICAL-MITIGATING COSMETIC COMPOSITIONS

(71) Applicant: ELC Management LLC, Melville, NY (US)

(72) Inventors: Jennifer S. HAGHPANAH, New York, NY (US); Robert Thomas MAIDHOF, JR., Kings Park, NY (US); Nadine PERNODET, Dix Hills, NY (US); Alison Deanne PAWLUS, Sunnyvale, CA (US); David Aaron VETTER, Locust Valley, NY (US)

(73) Assignee: ELC Management LLC, Melville, NY (US)

(21) Appl. No.: 19/085,178

(22) Filed: Mar. 20, 2025

(51) Int. Cl. A61K 8/9789; A61K 8/34; A61K 8/36; A61K 8/49; A61Q 17/00

(52) U.S. Cl. CPC A61K 8/9789; A61K 8/347; A61K 8/36; A61K 8/498; A61Q 17/00

(57) ABSTRACT

Inhibiting radical formation or quenching of radicals may aid in limiting damage to bodily structures resulting from exposure to these reactive species. Surprisingly, hydroxycinnamic acids, such as ferulic acid, may enhance the performance of polyphenols in inhibiting radical formation or quenching of radicals. Hydroxychromanes, such as copherol, may further improve the inhibition or quenching of radicals when combined with polyphenols and hydroxycinnamic acids. Cosmetic compositions having radical-mitigating properties may comprise at least one polyphenol, at least one hydroxychromane bearing at least one methyl group, and at least one hydroxycinnamic acid. The at least one polyphenol, the at least one hydroxychromane, and the at least one hydroxycinnamic acid may be present at or above a minimum concentration at which the synergistic interaction is first observable.

### FIELD

The present disclosure generally relates to cosmetic compositions, and more specifically, to cosmetic compositions that may mitigate the effects of free radicals in promoting skin damage or aging.

### BACKGROUND

The skin is a vital organ composed of several layers including the dermis, epidermis, and hypodermis, which covers the entire surface of the body and serves protective, sensation, immune, metabolic, and thermoregulatory functions. Like other organs, human skin is subject to aging and damage from external sources.

Skin is very sensitive to sunlight, artificial light, and pollutants. Prolonged exposure to one or more of these conditions may cause any of skin damage, redness, eruptions, skin burn, wrinkles, roughness, loss of stiffness, alteration of color or tone, inflammation, and the like. With increased awareness of the negative impacts of skin damage and similar conditions, cosmetic compositions and related dermatological products have been developed to convey functionality aimed at protecting human skin.

Exposure of the skin to free radicals (radicals) is one mechanism through which aging and damage of the skin may occur. Radicals are highly reactive molecular entities having one or more unpaired electrons. The high reactivity of radicals has the potential to lead to significant skin damage or damage to other bodily structures. Damage at a molecular level may result through the interaction of radicals with structures in the skin. For example, the interaction of radicals with cellular components may initiate oxidative chain reactions leading to degradation of biomolecules such as lipids, proteins, and DNA. Such oxidative damage may impair the ability of the skin to repair itself, accelerate the aging process, and contribute to loss of skin tone, which may impact the appearance of the skin, such as through development of wrinkles, fine lines, and hyperpigmentation, for instance.

As such, cosmetic compositions and similar dermatological products that may effectively mitigate the effects of radicals are highly desired.

### SUMMARY

In some embodiments, cosmetic compositions may comprise at least one polyphenol; at least one hydroxychromane bearing at least one methyl group; and at least one hydroxycinnamic acid.

In some or other embodiments, cosmetic compositions may comprise about 0.0005 wt % or above of at least one polyphenol; about 0.0005 wt % or above of at least one hydroxychromane bearing at least one methyl group; and about 0.0005 wt % or above of at least one hydroxycinnamic acid; wherein each wt % is based on total mass of the cosmetic composition.

In still other embodiments, methods may comprise applying a cosmetic composition of the present disclosure to a bodily structure; and inhibiting radical formation upon and/or within the bodily structure, and/or quenching radicals upon and/or within the bodily structure.

### BRIEF DESCRIPTION OF THE DRAWINGS

The following figures are included to illustrate certain aspects of the disclosure herein, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a bar graph showing the percent inhibition for formation of the ABTS radical cation for various samples.

FIG. 2 is a bar graph showing the percent inhibition for formation of the ABTS radical cation for various formulated samples.

FIG. 3 is a bar graph of spin counts as a percentage of DPPH spin counts for freshly prepared formulated samples and formulated samples aged for 3 months.

### DETAILED DESCRIPTION

The present disclosure generally relates to cosmetic compositions, and more specifically, to cosmetic compositions that may mitigate the effects of free radicals in promoting skin damage or aging.

As discussed above, limiting the effects of radicals upon the skin may be desirable due to the ability of these reactive species to promote skin damage and impact the appearance of skin. One approach that may be effective for suppressing the impact of radicals upon the skin is to apply cosmetic compositions or similar dermatological products containing one or more antiradical agents therein.

The present disclosure describes cosmetic compositions having enhanced radical-mitigating effects of antiradical compounds contained therein, as well as providing other related advantages. In particular, the present disclosure provides cosmetic compositions in which at least one hydroxycinnamic acid may interact with an antiradical compound at concentrations greater than or equal to those effective to impart radical inhibition or radical quenching effects that are more than additive compared to radical inhibition or radical quenching effect of the antiradical compound and the at least one hydroxycinnamic acid measured separately and at identical concentrations. That is, the antiradical compound and the at least one hydroxycinnamic acid may interact synergistically with one another in the course of exerting their radical-mitigating effects, wherein the concentration of the antiradical compound and the concentration of the at least one hydroxycinnamic acid are at or above a minimum concentration at which the synergistic interaction is first observable. Even more surprisingly, the synergistic interaction may be further enhanced by including at least one hydroxychromane bearing at least one methyl group in the cosmetic compositions.

In non-limiting examples, the antiradical compound in the cosmetic compositions described herein may comprise at least one polyphenol. Suitable polyphenols having antiradical properties are widely known and may include those contained in green tea extract, for example. Epigallocatechin gallate is an example polyphenol that may be found in green tea extract and that may be suitable for promoting radical inhibition and radical quenching using the cosmetic compositions described herein. Epigallocatechin gallate may be present alone as the antiradical compound or used in combination with other green tea polyphenols. Other polyphenols, alone or in combination with one or more green tea polyphenols, may also be suitable and are described further below.

By virtue of the synergistic interaction(s) afforded with hydroxychromanes and hydroxycinnamic acids, more effective radical-mitigating effects may be realized in cosmetic compositions containing polyphenols. The term “synergistic interaction” means that the amount of radical inhibition or radical quenching of a blend of components is more than additive compared to the radical inhibition or radical quenching performance of the components evaluated individually at identical concentrations. This benefit may allow cosmetic compositions to be formulated using lower amounts of polyphenols or other ingredients while still maintaining substantially equivalent antiradical efficacy, and/or affording more effective mitigation of radicals at a fixed concentration of polyphenols or other ingredients. Advantageously, lowering the amount(s) of polyphenols or other active components in cosmetic compositions may decrease raw material costs and lessen the risk of potential side effects that might occur at higher polyphenol concentrations.

Accordingly, cosmetic compositions of the present disclosure may comprise: at least one antiradical compound, such as at least one polyphenol; and at least one hydroxycinnamic acid, such as ferulic acid. The at least one polyphenol and the at least one hydroxycinnamic acid may be present at or above a minimum concentration where the at least one polyphenol and the at least one hydrocinnamic acid interact synergistically with one another. Preferably, the cosmetic compositions may further comprise at least one hydroxychromane bearing at least one methyl group, such as copherol, again at or above a minimum concentration at which the synergistic interaction occurs. As used herein, the term “copherol” refers to α-tocopherol or a blend of tocopherol isomers containing at least about 80 wt % α-tocopherol based on total mass. When the cosmetic compositions comprise at least one polyphenol, at least one hydroxychromane, and at least one hydroxycinnamic acid, a further increased synergistic interaction may be realized with respect to inhibiting radical formation or quenching existing radicals, as compared to cosmetic compositions lacking the at least one hydroxychromane.

In the present disclosure, the term “cosmetic composition” refers to any formulation that is applied to a bodily structure, such as a skin surface or hair, for achieving a specified purpose. The specified purpose is not considered to be particularly limited. The specified purpose may be to achieve a desired appearance or organoleptic effect, and to promote healthy skin or hair. Without being bound by theory or mechanism, the desired appearance or organoleptic effect may arise as a consequence of promoting radical mitigation upon the bodily structure. In non-limiting examples, the cosmetic compositions of the present disclosure may define a foundation, a skin conditioner, a skin serum, a lotion, or any combination thereof. Cosmetic compositions of the present disclosure may alter a user's appearance or be inconspicuous when worn. For example, dermatological products are a type of cosmetic composition that may be inconspicuous when worn.

Suitable polyphenols may include those that are present in green tea extract, for example. Catechins in green tea extract are known antioxidant compounds. Epigallocatechin gallate (EGCG) is the most abundant catechin in green tea extract. Other catechins present in significant quantities in green tea extract include epicatechin (EC), epicatechin gallate (ECG), and epigallocatechin (EGC). Any of the foregoing may be present in combination with one another in green tea extract suitable for use in the disclosure herein. Green tea extract may be used in the cosmetic compositions of the present disclosure without further refining the polyphenols therein. Some suitable green tea extracts may be further refined, however, to standardize the quantity of polyphenols present therein, such as about 40 wt % or above total polyphenols or about 60 wt % or above total polyphenols, or about 80 wt % or above total polyphenols, and/or to standardize the quantity of EGCG present therein, such as about 10 wt % or above or about 40 wt % or above, based on total mass of the green tea extract. For example, some suitable green tea extracts may include those containing about 90 wt % total polyphenols and at least about 44 wt % EGCG, or about 50 wt % total polyphenols and at least about 10 wt % EGCG. Alternately, any of the foregoing polyphenols or a combination thereof may be isolated from green tea extract in an at least partially purified form and utilized in the cosmetic compositions in the amounts described herein. Within green tea extract or as an isolate from green tea extract, any of the foregoing catechin polyphenols may be present in combination with other polyphenolic compounds commonly found in green tea extract, such as flavonoids and tannins, or other components of green tea extract such as caffeine, theobromine, theophylline, amino acids (e.g., L-theanine), and the like. Polyphenolic compounds from green tea extract may also be used in combination with polyphenols from other sources, such as those described subsequently.

Other polyphenols having antioxidant properties or extracts containing such alternative polyphenols may also be suitable for use in the cosmetic compositions disclosed herein. Suitable alternative polyphenols may include, for example, resveratrol (a stilbenoid polyphenol) or an extract containing resveratrol. Extracts containing resveratrol may include those of Vitis species such as grapes, Morus species such as mulberry, and several species within Polygonaceae family such as Reynoutria japonica (syn. Polygonum cuspidatum) and Reynoutria multiflora (syn. Polygonum multiflorum), commonly known as Japanese knotweed and fo-ti, respectively. Hydroxytyrosol is another example of a suitable polyphenol.

The at least one polyphenol (or a green tea extract or similar extract containing at least one polyphenol) may be present in various amounts in the cosmetic compositions described herein. The at least one polyphenol may be present in the cosmetic compositions in an amount of 0.0005 wt % or above, or 0.001 wt % or above, or 0.005 wt % or above, or 0.01 wt % or above, or 0.05 wt % or above, or 0.01 wt % or above, based on total mass of the cosmetic composition.

In non-limiting examples, the at least one polyphenol may be present in the cosmetic compositions in an amount ranging from about a1 to about a2 wt %, based on total mass of the cosmetic composition, wherein a1 and a2 are, independently, 0.0005, 0.0006, 0.0007, 0.0008, 0.0009, 0.001, 0.002, 0.003, 0.004, or 0.005, and where a1<a2. In non-limiting examples, the at least one polyphenol may be present in the cosmetic compositions in an amount ranging from about 0.0005 wt % to about 0.0006 wt %, about 0.0005 wt % to about 0.0007 wt %, about 0.0005 wt % to about 0.0008 wt %, about 0.0005 wt % to about 0.0009 wt %, about 0.0005 wt % to about 0.001 wt %, about 0.0005 wt % to about 0.002 wt %, about 0.0005 wt % to about 0.003 wt %, about 0.0005 wt % to about 0.004 wt %, about 0.0005 wt % to about 0.005 wt %, about 0.0006 wt % to about 0.0007 wt %, about 0.0006 wt % to about 0.0008 wt %, about 0.0006 wt % to about 0.0009 wt %, about 0.0006 wt % to about 0.001 wt %, about 0.0006 wt % to about 0.002 wt %, about 0.0006 wt % to about 0.003 wt %, about 0.0006 wt % to about 0.004 wt %, about 0.0006 wt % to about 0.005 wt %, about 0.0007 wt % to about 0.0008 wt %, about 0.0007 wt % to about 0.0009 wt %, about 0.0007 wt % to about 0.001 wt %, about 0.0007 wt % to about 0.002 wt %, about 0.0007 wt % to about 0.003 wt %, about 0.0007 wt % to about 0.004 wt %, about 0.0007 wt % to about 0.005 wt %, about 0.0008 wt % to about 0.0009 wt %, about 0.0008 wt % to about 0.001 wt %, about 0.0008 wt % to about 0.002 wt %, about 0.0008 wt % to about 0.003 wt %, about 0.0008 wt % to about 0.004 wt %, about 0.0008 wt % to about 0.005 wt %, about 0.0009 wt % to about 0.001 wt %, about 0.0009 wt % to about 0.002 wt %, about 0.0009 wt % to about 0.003 wt %, about 0.0005 wt % to about 0.004 wt %, about 0.0009 wt % to about 0.005 wt %, about 0.001 wt % to about 0.002 wt %, about 0.001 wt % to about 0.003 wt %, about 0.001 wt % to about 0.004 wt %, about 0.001 wt % to about 0.005 wt %, about 0.002 wt % to about 0.003 wt %, about 0.002 wt % to about 0.004 wt %, about 0.002 wt % to about 0.005 wt %, about 0.003 wt % to about 0.004 wt %, about 0.003 wt % to about 0.005 wt %, or about 0.004 wt % to about 0.005 wt %, based on total mass of the cosmetic composition. At least one catechin, either present in green tea extract or as an isolate, may be present in any of the foregoing amounts. Resveratrol and/or hydroxytyrosol may likewise be present in any of the foregoing amounts.

In some examples, the at least one polyphenol, such as at least one catechin, resveratrol, or hydroxytyrosol, may be present in the cosmetic compositions in an amount of about 0.0009 wt % to about 0.0011 wt %, based on total mass of the cosmetic compositions.

In other non-limiting examples, the at least one polyphenol may be present in the cosmetic compositions in an amount ranging from about a1 to about a2 wt %, based on total mass of the cosmetic composition, wherein a1 and a2 are, independently, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, or 0.5 and where a1<a2. In non-limiting examples, the at least one polyphenol may be present in the cosmetic compositions in an amount ranging from about 0.005 wt % to about 0.006 wt %, about 0.005 wt % to about 0.007 wt %, about 0.005 wt % to about 0.008 wt %, about 0.005 wt % to about 0.009 wt %, about 0.005 wt % to about 0.01 wt %, about 0.005 wt % to about 0.02 wt %, about 0.005 wt % to about 0.03 wt %, about 0.005 wt % to about 0.04 wt %, about 0.005 wt % to about 0.05 wt %, about 0.005 wt % to about 0.06 wt %, about 0.005 wt % to about 0.07 wt %, about 0.005 wt % to about 0.08 wt %, about 0.005 wt % to about 0.09 wt %, about 0.005 wt % to about 0.1 wt %, about 0.005 wt % to about 0.2 wt %, about 0.005 wt % to about 0.3 wt %, about 0.005 wt % to about 0.4 wt %, about 0.005 wt % to about 0.5 wt %, about 0.006 wt % to about 0.007 wt %, about 0.006 wt % to about 0.008 wt %, about 0.006 wt % to about 0.009 wt %, about 0.006 wt % to about 0.01 wt %, about 0.006 wt % to about 0.02 wt %, about 0.006 wt % to about 0.03 wt %, about 0.006 wt % to about 0.04 wt %, about 0.006 wt % to about 0.05 wt %, about 0.006 wt % to about 0.06 wt %, about 0.006 wt % to about 0.07 wt %, about 0.006 wt % to about 0.08 wt %, about 0.006 wt % to about 0.09 wt %, about 0.006 wt % to about 0.1 wt %, about 0.006 wt % to about 0.2 wt %, about 0.006 wt % to about 0.3 wt %, about 0.006 wt % to about 0.4 wt %, about 0.006 wt % to about 0.5 wt %, about 0.007 wt % to about 0.008 wt %, about 0.007 wt % to about 0.009 wt %, about 0.007 wt % to about 0.01 wt %, about 0.007 wt % to about 0.02 wt %, about 0.007 wt % to about 0.03 wt %, about 0.007 wt % to about 0.04 wt %, about 0.007 wt % to about 0.05 wt %, about 0.007 wt % to about 0.06 wt %, about 0.007 wt % to about 0.07 wt %, about 0.007 wt % to about 0.08 wt %, about 0.007 wt % to about 0.09 wt %, about 0.007 wt % to about 0.1 wt %, about 0.007 wt % to about 0.2 wt %, about 0.007 wt % to about 0.3 wt %, about 0.007 wt % to about 0.4 wt %, about 0.007 wt % to about 0.5 wt %, about 0.008 wt % to about 0.009 wt %, about 0.008 wt % to about 0.01 wt %, about 0.008 wt % to about 0.02 wt %, about 0.008 wt % to about 0.03 wt %, about 0.008 wt % to about 0.04 wt %, about 0.008 wt % to about 0.05 wt %, about 0.008 wt % to about 0.06 wt %, about 0.008 wt % to about 0.07 wt %, about 0.008 wt % to about 0.08 wt %, about 0.008 wt % to about 0.09 wt %, about 0.008 wt % to about 0.1 wt %, about 0.008 wt % to about 0.2 wt %, about 0.008 wt % to about 0.3 wt %, about 0.008 wt % to about 0.4 wt %, about 0.008 wt % to about 0.5 wt %, about 0.009 wt % to about 0.01 wt %, about 0.009 wt % to about 0.02 wt %, about 0.009 wt % to about 0.03 wt %, about 0.009 wt % to about 0.04 wt %, about 0.009 wt % to about 0.05 wt %, about 0.009 wt % to about 0.06 wt %, about 0.009 wt % to about 0.07 wt %, about 0.009 wt % to about 0.08 wt %, about 0.009 wt % to about 0.09 wt %, about 0.009 wt % to about 0.1 wt %, about 0.009 wt % to about 0.2 wt %, about 0.009 wt % to about 0.3 wt %, about 0.009 wt % to about 0.4 wt %, about 0.009 wt % to about 0.5 wt %, about 0.01 wt % to about 0.02 wt %, about 0.01 wt % to about 0.03 wt %, about 0.01 wt % to about 0.04 wt %, about 0.01 wt % to about 0.05 wt %, about 0.01 wt % to about 0.06 wt %, about 0.01 wt % to about 0.07 wt %, about 0.01 wt % to about 0.08 wt %, about 0.01 wt % to about 0.09 wt %, about 0.01 wt % to about 0.1 wt %, about 0.01 wt % to about 0.2 wt %, about 0.01 wt % to about 0.3 wt %, about 0.01 wt % to about 0.4 wt %, about 0.01 wt % to about 0.5 wt %, about 0.01 wt % to about 0.03 wt %, about 0.01 wt % to about 0.04 wt %, about 0.01 wt % to about 0.05 wt %, about 0.01 wt % to about 0.06 wt %, about 0.01 wt % to about 0.07 wt %, about 0.01 wt % to about 0.08 wt %, about 0.01 wt % to about 0.09 wt %, about 0.01 wt % to about 0.1 wt %, about 0.01 wt % to about 0.2 wt %, about 0.01 wt % to about 0.3 wt %, about 0.01 wt % to about 0.4 wt %, about 0.01 wt % to about 0.5 wt %, about 0.02 wt % to about 0.03 wt %, about 0.02 wt % to about 0.04 wt %, about 0.02 wt % to about 0.05 wt %, about 0.02 wt % to about 0.06 wt %, about 0.02 wt % to about 0.07 wt %, about 0.02 wt % to about 0.08 wt %, about 0.02 wt % to about 0.09 wt %, about 0.02 wt % to about 0.1 wt %, about 0.02 wt % to about 0.2 wt %, about 0.02 wt % to about 0.3 wt %, about 0.02 wt % to about 0.4 wt %, about 0.02 wt % to about 0.5 wt %, about 0.03 wt % to about 0.04 wt %, about 0.03 wt % to about 0.05 wt %, about 0.03 wt % to about 0.06 wt %, about 0.03 wt % to about 0.07 wt %, about 0.03 wt % to about 0.08 wt %, about 0.03 wt % to about 0.09 wt %, about 0.03 wt % to about 0.1 wt %, about 0.03 wt % to about 0.2 wt %, about 0.03 wt % to about 0.3 wt %, about 0.03 wt % to about 0.4 wt %, about 0.03 wt % to about 0.5 wt %, about 0.04 wt % to about 0.05 wt %, about 0.04 wt % to about 0.06 wt %, about 0.04 wt % to about 0.07 wt %, about 0.04 wt % to about 0.08 wt %, about 0.04 wt % to about 0.09 wt %, about 0.04 wt % to about 0.1 wt %, about 0.04 wt % to about 0.2 wt %, about 0.04 wt % to about 0.3 wt %, about 0.04 wt % to about 0.4 wt %, about 0.04 wt % to about 0.5 wt %, about 0.05 wt % to about 0.06 wt %, about 0.05 wt % to about 0.07 wt %, about 0.05 wt % to about 0.08 wt %, about 0.05 wt % to about 0.09 wt %, about 0.05 wt % to about 0.1 wt %, about 0.05 wt % to about 0.2 wt %, about 0.05 wt % to about 0.3 wt %, about 0.05 wt % to about 0.4 wt %, about 0.05 wt % to about 0.5 wt %, about 0.06 wt % to about 0.07 wt %, about 0.06 wt % to about 0.08 wt %, about 0.06 wt % to about 0.09 wt %, about 0.06 wt % to about 0.1 wt %, about 0.06 wt % to about 0.2 wt %, about 0.06 wt % to about 0.3 wt %, about 0.06 wt % to about 0.4 wt %, about 0.06 wt % to about 0.5 wt %, about 0.07 wt % to about 0.08 wt %, about 0.07 wt % to about 0.09 wt %, about 0.07 wt % to about 0.1 wt %, about 0.07 wt % to about 0.2 wt %, about 0.07 wt % to about 0.3 wt %, about 0.07 wt % to about 0.4 wt %, about 0.07 wt % to about 0.5 wt %, about 0.08 wt % to about 0.09 wt %, about 0.08 wt % to about 0.1 wt %, about 0.08 wt % to about 0.2 wt %, about 0.08 wt % to about 0.3 wt %, about 0.08 wt % to about 0.4 wt %, about 0.08 wt % to about 0.5 wt %, about 0.09 wt % to about 0.1 wt %, about 0.09 wt % to about 0.2 wt %, about 0.09 wt % to about 0.3 wt %, about 0.09 wt % to about 0.4 wt %, about 0.09 wt % to about 0.5 wt %, about 0.1 wt % to about 0.2 wt %, about 0.1 wt % to about 0.3 wt %, about 0.1 wt % to about 0.4 wt %, about 0.1 wt % to about 0.5 wt %, about 0.2 wt % to about 0.3 wt %, about 0.2 wt % to about 0.4 wt %, about 0.2 wt % to about 0.5 wt %, about 0.3 wt % to about 0.4 wt %, about 0.3 wt % to about 0.5 wt %, or about 0.4 wt % to about 0.5 wt %, based on total mass of the cosmetic composition. At least one catechin, either present in green tea extract or as an isolate, may be present in any of the foregoing amounts. Resveratrol and/or hydroxytyrosol may likewise be present in any of the foregoing amounts.

Suitable hydroxychromanes include those bearing with at least one methyl group. Such hydroxychromanes may be synthesized or isolated from a suitable natural source. In some examples, the at least one hydroxychromane may comprise copherol.

The at least one hydroxychromane, such as copherol, may be present in various amounts in the cosmetic compositions described herein. The at least one hydroxychromane may be present in the cosmetic compositions in an amount of 0.0005 wt % or above, or 0.001 wt % or above, or 0.005 wt % or above, or 0.01 wt % or above, or 0.05 wt % or above, or 0.01 wt % or above, based on total mass of the cosmetic composition.

In non-limiting examples, the at least one hydroxychromane may be present in the cosmetic compositions in an amount ranging from about b1 to about b2 wt %, based on total mass of the cosmetic composition, wherein b1 and b2 are, independently, 0.0005, 0.0006, 0.0007, 0.0008, 0.0009, 0.001, 0.002, 0.003, 0.004, or 0.005, and where b1<b2. In non-limiting examples, the at least one hydroxychromane may be present in the cosmetic compositions in an amount ranging from about 0.0005 wt % to about 0.0006 wt %, about 0.0005 wt % to about 0.0007 wt %, about 0.0005 wt % to about 0.0008 wt %, about 0.0005 wt % to about 0.0009 wt %, about 0.0005 wt % to about 0.001 wt %, about 0.0005 wt % to about 0.002 wt %, about 0.0005 wt % to about 0.003 wt %, about 0.0005 wt % to about 0.004 wt %, about 0.0005 wt % to about 0.005 wt %, about 0.0006 wt % to about 0.0007 wt %, about 0.0006 wt % to about 0.0008 wt %, about 0.0006 wt % to about 0.0009 wt %, about 0.0006 wt % to about 0.001 wt %, about 0.0006 wt % to about 0.002 wt %, about 0.0006 wt % to about 0.003 wt %, about 0.0006 wt % to about 0.004 wt %, about 0.0006 wt % to about 0.005 wt %, about 0.0007 wt % to about 0.0008 wt %, about 0.0007 wt % to about 0.0009 wt %, about 0.0007 wt % to about 0.001 wt %, about 0.0007 wt % to about 0.002 wt %, about 0.0007 wt % to about 0.003 wt %, about 0.0007 wt % to about 0.004 wt %, about 0.0007 wt % to about 0.005 wt %, about 0.0008 wt % to about 0.0009 wt %, about 0.0008 wt % to about 0.001 wt %, about 0.0008 wt % to about 0.002 wt %, about 0.0008 wt % to about 0.003 wt %, about 0.0008 wt % to about 0.004 wt %, about 0.0008 wt % to about 0.005 wt %, about 0.0009 wt % to about 0.001 wt %, about 0.0009 wt % to about 0.002 wt %, about 0.0009 wt % to about 0.003 wt %, about 0.0005 wt % to about 0.004 wt %, about 0.0009 wt % to about 0.005 wt %, about 0.001 wt % to about 0.002 wt %, about 0.001 wt % to about 0.003 wt %, about 0.001 wt % to about 0.004 wt %, about 0.001 wt % to about 0.005 wt %, about 0.002 wt % to about 0.003 wt %, about 0.002 wt % to about 0.004 wt %, about 0.002 wt % to about 0.005 wt %, about 0.003 wt % to about 0.004 wt %, about 0.003 wt % to about 0.005 wt %, or about 0.004 wt % to about 0.005 wt %, based on total mass of the cosmetic composition.

In some examples, the at least one hydroxychromane, such as copherol, may be present in the cosmetic compositions in an amount of about 0.0009 wt % to about 0.0011 wt %, based on total mass of the cosmetic compositions.

In other non-limiting examples, the at least one hydroxychromane, such as copherol, may be present in the cosmetic compositions in an amount ranging from about b1 to about b2 wt %, based on total mass of the cosmetic composition, wherein b1 and b2 are, independently, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, or 0.5 and where b1<b2. In non-limiting examples, the at least one hydroxychromane, such as copherol, may be present in the cosmetic compositions in an amount ranging from about 0.005 wt % to about 0.006 wt %, about 0.005 wt % to about 0.007 wt %, about 0.005 wt % to about 0.008 wt %, about 0.005 wt % to about 0.009 wt %, about 0.005 wt % to about 0.01 wt %, about 0.005 wt % to about 0.02 wt %, about 0.005 wt % to about 0.03 wt %, about 0.005 wt % to about 0.04 wt %, about 0.005 wt % to about 0.05 wt %, about 0.005 wt % to about 0.06 wt %, about 0.005 wt % to about 0.07 wt %, about 0.005 wt % to about 0.08 wt %, about 0.005 wt % to about 0.09 wt %, about 0.005 wt % to about 0.1 wt %, about 0.005 wt % to about 0.2 wt %, about 0.005 wt % to about 0.3 wt %, about 0.005 wt % to about 0.4 wt %, about 0.005 wt % to about 0.5 wt %, about 0.006 wt % to about 0.007 wt %, about 0.006 wt % to about 0.008 wt %, about 0.006 wt % to about 0.009 wt %, about 0.006 wt % to about 0.01 wt %, about 0.006 wt % to about 0.02 wt %, about 0.006 wt % to about 0.03 wt %, about 0.006 wt % to about 0.04 wt %, about 0.006 wt % to about 0.05 wt %, about 0.006 wt % to about 0.06 wt %, about 0.006 wt % to about 0.07 wt %, about 0.006 wt % to about 0.08 wt %, about 0.006 wt % to about 0.09 wt %, about 0.006 wt % to about 0.1 wt %, about 0.006 wt % to about 0.2 wt %, about 0.006 wt % to about 0.3 wt %, about 0.006 wt % to about 0.4 wt %, about 0.006 wt % to about 0.5 wt %, about 0.007 wt % to about 0.008 wt %, about 0.007 wt % to about 0.009 wt %, about 0.007 wt % to about 0.01 wt %, about 0.007 wt % to about 0.02 wt %, about 0.007 wt % to about 0.03 wt %, about 0.007 wt % to about 0.04 wt %, about 0.007 wt % to about 0.05 wt %, about 0.007 wt % to about 0.06 wt %, about 0.007 wt % to about 0.07 wt %, about 0.007 wt % to about 0.08 wt %, about 0.007 wt % to about 0.09 wt %, about 0.007 wt % to about 0.1 wt %, about 0.007 wt % to about 0.2 wt %, about 0.007 wt % to about 0.3 wt %, about 0.007 wt % to about 0.4 wt %, about 0.007 wt % to about 0.5 wt %, about 0.008 wt % to about 0.009 wt %, about 0.008 wt % to about 0.01 wt %, about 0.008 wt % to about 0.02 wt %, about 0.008 wt % to about 0.03 wt %, about 0.008 wt % to about 0.04 wt %, about 0.008 wt % to about 0.05 wt %, about 0.008 wt % to about 0.06 wt %, about 0.008 wt % to about 0.07 wt %, about 0.008 wt % to about 0.08 wt %, about 0.008 wt % to about 0.09 wt %, about 0.008 wt % to about 0.1 wt %, about 0.008 wt % to about 0.2 wt %, about 0.008 wt % to about 0.3 wt %, about 0.008 wt % to about 0.4 wt %, about 0.008 wt % to about 0.5 wt %, about 0.009 wt % to about 0.01 wt %, about 0.009 wt % to about 0.02 wt %, about 0.009 wt % to about 0.03 wt %, about 0.009 wt % to about 0.04 wt %, about 0.009 wt % to about 0.05 wt %, about 0.009 wt % to about 0.06 wt %, about 0.009 wt % to about 0.07 wt %, about 0.009 wt % to about 0.08 wt %, about 0.009 wt % to about 0.09 wt %, about 0.009 wt % to about 0.1 wt %, about 0.009 wt % to about 0.2 wt %, about 0.009 wt % to about 0.3 wt %, about 0.009 wt % to about 0.4 wt %, about 0.009 wt % to about 0.5 wt %, about 0.01 wt % to about 0.02 wt %, about 0.01 wt % to about 0.03 wt %, about 0.01 wt % to about 0.04 wt %, about 0.01 wt % to about 0.05 wt %, about 0.01 wt % to about 0.06 wt %, about 0.01 wt % to about 0.07 wt %, about 0.01 wt % to about 0.08 wt %, about 0.01 wt % to about 0.09 wt %, about 0.01 wt % to about 0.1 wt %, about 0.01 wt % to about 0.2 wt %, about 0.01 wt % to about 0.3 wt %, about 0.01 wt % to about 0.4 wt %, about 0.01 wt % to about 0.5 wt %, about 0.01 wt % to about 0.03 wt %, about 0.01 wt % to about 0.04 wt %, about 0.01 wt % to about 0.05 wt %, about 0.01 wt % to about 0.06 wt %, about 0.01 wt % to about 0.07 wt %, about 0.01 wt % to about 0.08 wt %, about 0.01 wt % to about 0.09 wt %, about 0.01 wt % to about 0.1 wt %, about 0.01 wt % to about 0.2 wt %, about 0.01 wt % to about 0.3 wt %, about 0.01 wt % to about 0.4 wt %, about 0.01 wt % to about 0.5 wt %, about 0.02 wt % to about 0.03 wt %, about 0.02 wt % to about 0.04 wt %, about 0.02 wt % to about 0.05 wt %, about 0.02 wt % to about 0.06 wt %, about 0.02 wt % to about 0.07 wt %, about 0.02 wt % to about 0.08 wt %, about 0.02 wt % to about 0.09 wt %, about 0.02 wt % to about 0.1 wt %, about 0.02 wt % to about 0.2 wt %, about 0.02 wt % to about 0.3 wt %, about 0.02 wt % to about 0.4 wt %, about 0.02 wt % to about 0.5 wt %, about 0.03 wt % to about 0.04 wt %, about 0.03 wt % to about 0.05 wt %, about 0.03 wt % to about 0.06 wt %, about 0.03 wt % to about 0.07 wt %, about 0.03 wt % to about 0.08 wt %, about 0.03 wt % to about 0.09 wt %, about 0.03 wt % to about 0.1 wt %, about 0.03 wt % to about 0.2 wt %, about 0.03 wt % to about 0.3 wt %, about 0.03 wt % to about 0.4 wt %, about 0.03 wt % to about 0.5 wt %, about 0.04 wt % to about 0.05 wt %, about 0.04 wt % to about 0.06 wt %, about 0.04 wt % to about 0.07 wt %, about 0.04 wt % to about 0.08 wt %, about 0.04 wt % to about 0.09 wt %, about 0.04 wt % to about 0.1 wt %, about 0.04 wt % to about 0.2 wt %, about 0.04 wt % to about 0.3 wt %, about 0.04 wt % to about 0.4 wt %, about 0.04 wt % to about 0.5 wt %, about 0.05 wt % to about 0.06 wt %, about 0.05 wt % to about 0.07 wt %, about 0.05 wt % to about 0.08 wt %, about 0.05 wt % to about 0.09 wt %, about 0.05 wt % to about 0.1 wt %, about 0.05 wt % to about 0.2 wt %, about 0.05 wt % to about 0.3 wt %, about 0.05 wt % to about 0.4 wt %, about 0.05 wt % to about 0.5 wt %, about 0.06 wt % to about 0.07 wt %, about 0.06 wt % to about 0.08 wt %, about 0.06 wt % to about 0.09 wt %, about 0.06 wt % to about 0.1 wt %, about 0.06 wt % to about 0.2 wt %, about 0.06 wt % to about 0.3 wt %, about 0.06 wt % to about 0.4 wt %, about 0.06 wt % to about 0.5 wt %, about 0.07 wt % to about 0.08 wt %, about 0.07 wt % to about 0.09 wt %, about 0.07 wt % to about 0.1 wt %, about 0.07 wt % to about 0.2 wt %, about 0.07 wt % to about 0.3 wt %, about 0.07 wt % to about 0.4 wt %, about 0.07 wt % to about 0.5 wt %, about 0.08 wt % to about 0.09 wt %, about 0.08 wt % to about 0.1 wt %, about 0.08 wt % to about 0.2 wt %, about 0.08 wt % to about 0.3 wt %, about 0.08 wt % to about 0.4 wt %, about 0.08 wt % to about 0.5 wt %, about 0.09 wt % to about 0.1 wt %, about 0.09 wt % to about 0.2 wt %, about 0.09 wt % to about 0.3 wt %, about 0.09 wt % to about 0.4 wt %, about 0.09 wt % to about 0.5 wt %, about 0.1 wt % to about 0.2 wt %, about 0.1 wt % to about 0.3 wt %, about 0.1 wt % to about 0.4 wt %, about 0.1 wt % to about 0.5 wt %, about 0.2 wt % to about 0.3 wt %, about 0.2 wt % to about 0.4 wt %, about 0.2 wt % to about 0.5 wt %, about 0.3 wt % to about 0.4 wt %, about 0.3 wt % to about 0.5 wt %, or about 0.4 wt % to about 0.5 wt %, based on total mass of the cosmetic composition.

Hydroxycinnamic acids contain a carboxylic acid group linked to an aromatic ring via a trans-double bond. The aromatic ring is substituted with at least one phenolic hydroxyl group and optionally, at least one alkoxy group. Examples of hydroxycinnamic acids that may be suitable for use in the disclosure herein include, for example, caffeic acid, chicoric acid, cinnamic acid, chlorogenic acid, ferulic acid, ferulic acid dimers, coumaric acid, sinapinic acid, and the like. Ferulic acid may be a particularly suitable hydroxycinnamic acid for use in the cosmetic compositions of the present disclosure.

The at least one hydroxycinnamic acid, such as ferulic acid, may be present in various amounts in the cosmetic compositions described herein. The at least one hydroxycinnamic acid may be present in the cosmetic compositions in an amount of 0.0005 wt % or above, or 0.001 wt % or above, or 0.005 wt % or above, or 0.01 wt % or above, or 0.05 wt % or above, or 0.01 wt % or above, based on total mass of the cosmetic composition.

In non-limiting examples, the at least one hydroxycinnamic acid may be present in the cosmetic compositions in an amount ranging from about c1 to about c2 wt %, based on total mass of the cosmetic composition, wherein c1 and c2 are, independently, 0.0005, 0.0006, 0.0007, 0.0008, 0.0009, 0.001, 0.002, 0.003, 0.004, or 0.005, and where c1<c2. In non-limiting examples, the at least one hydroxycinnamic acid may be present in the cosmetic compositions in an amount ranging from about 0.0005 wt % to about 0.0006 wt %, about 0.0005 wt % to about 0.0007 wt %, about 0.0005 wt % to about 0.0008 wt %, about 0.0005 wt % to about 0.0009 wt %, about 0.0005 wt % to about 0.001 wt %, about 0.0005 wt % to about 0.002 wt %, about 0.0005 wt % to about 0.003 wt %, about 0.0005 wt % to about 0.004 wt %, about 0.0005 wt % to about 0.005 wt %, about 0.0006 wt % to about 0.0007 wt %, about 0.0006 wt % to about 0.0008 wt %, about 0.0006 wt % to about 0.0009 wt %, about 0.0006 wt % to about 0.001 wt %, about 0.0006 wt % to about 0.002 wt %, about 0.0006 wt % to about 0.003 wt %, about 0.0006 wt % to about 0.004 wt %, about 0.0006 wt % to about 0.005 wt %, about 0.0007 wt % to about 0.0008 wt %, about 0.0007 wt % to about 0.0009 wt %, about 0.0007 wt % to about 0.001 wt %, about 0.0007 wt % to about 0.002 wt %, about 0.0007 wt % to about 0.003 wt %, about 0.0007 wt % to about 0.004 wt %, about 0.0007 wt % to about 0.005 wt %, about 0.0008 wt % to about 0.0009 wt %, about 0.0008 wt % to about 0.001 wt %, about 0.0008 wt % to about 0.002 wt %, about 0.0008 wt % to about 0.003 wt %, about 0.0008 wt % to about 0.004 wt %, about 0.0008 wt % to about 0.005 wt %, about 0.0009 wt % to about 0.001 wt %, about 0.0009 wt % to about 0.002 wt %, about 0.0009 wt % to about 0.003 wt %, about 0.0005 wt % to about 0.004 wt %, about 0.0009 wt % to about 0.005 wt %, about 0.001 wt % to about 0.002 wt %, about 0.001 wt % to about 0.003 wt %, about 0.001 wt % to about 0.004 wt %, about 0.001 wt % to about 0.005 wt %, about 0.002 wt % to about 0.003 wt %, about 0.002 wt % to about 0.004 wt %, about 0.002 wt % to about 0.005 wt %, about 0.003 wt % to about 0.004 wt %, about 0.003 wt % to about 0.005 wt %, or about 0.004 wt % to about 0.005 wt %, based on total mass of the cosmetic composition.

In some examples, the at least one hydroxycinnamic acid, such as ferulic acid, may be present in the cosmetic compositions in an amount of about 0.0009 wt % to about 0.0011 wt %, based on total mass of the cosmetic compositions.

In other non-limiting examples, the at least one hydroxycinnamic acid, such as ferulic acid, may be present in the cosmetic compositions in an amount ranging from about c1 to about c2 wt %, based on total mass of the cosmetic composition, wherein c1 and c2 are, independently, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, or 0.5 and where c1<c2. In non-limiting examples, the at least one hydroxycinnamic acid, such as ferulic acid, may be present in the cosmetic compositions in an amount ranging from about 0.005 wt % to about 0.006 wt %, about 0.005 wt % to about 0.007 wt %, about 0.005 wt % to about 0.008 wt %, about 0.005 wt % to about 0.009 wt %, about 0.005 wt % to about 0.01 wt %, about 0.005 wt % to about 0.02 wt %, about 0.005 wt % to about 0.03 wt %, about 0.005 wt % to about 0.04 wt %, about 0.005 wt % to about 0.05 wt %, about 0.005 wt % to about 0.06 wt %, about 0.005 wt % to about 0.07 wt %, about 0.005 wt % to about 0.08 wt %, about 0.005 wt % to about 0.09 wt %, about 0.005 wt % to about 0.1 wt %, about 0.005 wt % to about 0.2 wt %, about 0.005 wt % to about 0.3 wt %, about 0.005 wt % to about 0.4 wt %, about 0.005 wt % to about 0.5 wt %, about 0.006 wt % to about 0.007 wt %, about 0.006 wt % to about 0.008 wt %, about 0.006 wt % to about 0.009 wt %, about 0.006 wt % to about 0.01 wt %, about 0.006 wt % to about 0.02 wt %, about 0.006 wt % to about 0.03 wt %, about 0.006 wt % to about 0.04 wt %, about 0.006 wt % to about 0.05 wt %, about 0.006 wt % to about 0.06 wt %, about 0.006 wt % to about 0.07 wt %, about 0.006 wt % to about 0.08 wt %, about 0.006 wt % to about 0.09 wt %, about 0.006 wt % to about 0.1 wt %, about 0.006 wt % to about 0.2 wt %, about 0.006 wt % to about 0.3 wt %, about 0.006 wt % to about 0.4 wt %, about 0.006 wt % to about 0.5 wt %, about 0.007 wt % to about 0.008 wt %, about 0.007 wt % to about 0.009 wt %, about 0.007 wt % to about 0.01 wt %, about 0.007 wt % to about 0.02 wt %, about 0.007 wt % to about 0.03 wt %, about 0.007 wt % to about 0.04 wt %, about 0.007 wt % to about 0.05 wt %, about 0.007 wt % to about 0.06 wt %, about 0.007 wt % to about 0.07 wt %, about 0.007 wt % to about 0.08 wt %, about 0.007 wt % to about 0.09 wt %, about 0.007 wt % to about 0.1 wt %, about 0.007 wt % to about 0.2 wt %, about 0.007 wt % to about 0.3 wt %, about 0.007 wt % to about 0.4 wt %, about 0.007 wt % to about 0.5 wt %, about 0.008 wt % to about 0.009 wt %, about 0.008 wt % to about 0.01 wt %, about 0.008 wt % to about 0.02 wt %, about 0.008 wt % to about 0.03 wt %, about 0.008 wt % to about 0.04 wt %, about 0.008 wt % to about 0.05 wt %, about 0.008 wt % to about 0.06 wt %, about 0.008 wt % to about 0.07 wt %, about 0.008 wt % to about 0.08 wt %, about 0.008 wt % to about 0.09 wt %, about 0.008 wt % to about 0.1 wt %, about 0.008 wt % to about 0.2 wt %, about 0.008 wt % to about 0.3 wt %, about 0.008 wt % to about 0.4 wt %, about 0.008 wt % to about 0.5 wt %, about 0.009 wt % to about 0.01 wt %, about 0.009 wt % to about 0.02 wt %, about 0.009 wt % to about 0.03 wt %, about 0.009 wt % to about 0.04 wt %, about 0.009 wt % to about 0.05 wt %, about 0.009 wt % to about 0.06 wt %, about 0.009 wt % to about 0.07 wt %, about 0.009 wt % to about 0.08 wt %, about 0.009 wt % to about 0.09 wt %, about 0.009 wt % to about 0.1 wt %, about 0.009 wt % to about 0.2 wt %, about 0.009 wt % to about 0.3 wt %, about 0.009 wt % to about 0.4 wt %, about 0.009 wt % to about 0.5 wt %, about 0.01 wt % to about 0.02 wt %, about 0.01 wt % to about 0.03 wt %, about 0.01 wt % to about 0.04 wt %, about 0.01 wt % to about 0.05 wt %, about 0.01 wt % to about 0.06 wt %, about 0.01 wt % to about 0.07 wt %, about 0.01 wt % to about 0.08 wt %, about 0.01 wt % to about 0.09 wt %, about 0.01 wt % to about 0.1 wt %, about 0.01 wt % to about 0.2 wt %, about 0.01 wt % to about 0.3 wt %, about 0.01 wt % to about 0.4 wt %, about 0.01 wt % to about 0.5 wt %, about 0.01 wt % to about 0.03 wt %, about 0.01 wt % to about 0.04 wt %, about 0.01 wt % to about 0.05 wt %, about 0.01 wt % to about 0.06 wt %, about 0.01 wt % to about 0.07 wt %, about 0.01 wt % to about 0.08 wt %, about 0.01 wt % to about 0.09 wt %, about 0.01 wt % to about 0.1 wt %, about 0.01 wt % to about 0.2 wt %, about 0.01 wt % to about 0.3 wt %, about 0.01 wt % to about 0.4 wt %, about 0.01 wt % to about 0.5 wt %, about 0.02 wt % to about 0.03 wt %, about 0.02 wt % to about 0.04 wt %, about 0.02 wt % to about 0.05 wt %, about 0.02 wt % to about 0.06 wt %, about 0.02 wt % to about 0.07 wt %, about 0.02 wt % to about 0.08 wt %, about 0.02 wt % to about 0.09 wt %, about 0.02 wt % to about 0.1 wt %, about 0.02 wt % to about 0.2 wt %, about 0.02 wt % to about 0.3 wt %, about 0.02 wt % to about 0.4 wt %, about 0.02 wt % to about 0.5 wt %, about 0.03 wt % to about 0.04 wt %, about 0.03 wt % to about 0.05 wt %, about 0.03 wt % to about 0.06 wt %, about 0.03 wt % to about 0.07 wt %, about 0.03 wt % to about 0.08 wt %, about 0.03 wt % to about 0.09 wt %, about 0.03 wt % to about 0.1 wt %, about 0.03 wt % to about 0.2 wt %, about 0.03 wt % to about 0.3 wt %, about 0.03 wt % to about 0.4 wt %, about 0.03 wt % to about 0.5 wt %, about 0.04 wt % to about 0.05 wt %, about 0.04 wt % to about 0.06 wt %, about 0.04 wt % to about 0.07 wt %, about 0.04 wt % to about 0.08 wt %, about 0.04 wt % to about 0.09 wt %, about 0.04 wt % to about 0.1 wt %, about 0.04 wt % to about 0.2 wt %, about 0.04 wt % to about 0.3 wt %, about 0.04 wt % to about 0.4 wt %, about 0.04 wt % to about 0.5 wt %, about 0.05 wt % to about 0.06 wt %, about 0.05 wt % to about 0.07 wt %, about 0.05 wt % to about 0.08 wt %, about 0.05 wt % to about 0.09 wt %, about 0.05 wt % to about 0.1 wt %, about 0.05 wt % to about 0.2 wt %, about 0.05 wt % to about 0.3 wt %, about 0.05 wt % to about 0.4 wt %, about 0.05 wt % to about 0.5 wt %, about 0.06 wt % to about 0.07 wt %, about 0.06 wt % to about 0.08 wt %, about 0.06 wt % to about 0.09 wt %, about 0.06 wt % to about 0.1 wt %, about 0.06 wt % to about 0.2 wt %, about 0.06 wt % to about 0.3 wt %, about 0.06 wt % to about 0.4 wt %, about 0.06 wt % to about 0.5 wt %, about 0.07 wt % to about 0.08 wt %, about 0.07 wt % to about 0.09 wt %, about 0.07 wt % to about 0.1 wt %, about 0.07 wt % to about 0.2 wt %, about 0.07 wt % to about 0.3 wt %, about 0.07 wt % to about 0.4 wt %, about 0.07 wt % to about 0.5 wt %, about 0.08 wt % to about 0.09 wt %, about 0.08 wt % to about 0.1 wt %, about 0.08 wt % to about 0.2 wt %, about 0.08 wt % to about 0.3 wt %, about 0.08 wt % to about 0.4 wt %, about 0.08 wt % to about 0.5 wt %, about 0.09 wt % to about 0.1 wt %, about 0.09 wt % to about 0.2 wt %, about 0.09 wt % to about 0.3 wt %, about 0.09 wt % to about 0.4 wt %, about 0.09 wt % to about 0.5 wt %, about 0.1 wt % to about 0.2 wt %, about 0.1 wt % to about 0.3 wt %, about 0.1 wt % to about 0.4 wt %, about 0.1 wt % to about 0.5 wt %, about 0.2 wt % to about 0.3 wt %, about 0.2 wt % to about 0.4 wt %, about 0.2 wt % to about 0.5 wt %, about 0.3 wt % to about 0.4 wt %, about 0.3 wt % to about 0.5 wt %, or about 0.4 wt % to about 0.5 wt %, based on total mass of the cosmetic composition.

The at least one polyphenol, the at least one hydroxychromane, and the hydroxycinnamic acid may be present in equal amounts or at varying ratios with respect to one another in the cosmetic compositions described herein. In some examples, the at least one polyphenol and the at least one hydroxychromane may be present at a mass ratio ranging from about 1:20 to about 20:1, or about 1:10 to about 10:1, or about 1:5 to about 5:1, or about 1:2 to about 2:1. In some examples, the at least one polyphenol and the at least one hydroxycinnamic acid may be present at a mass ratio ranging from about 1:20 to about 20:1, or about 1:10 to about 10:1, 1:5 to about 5:1, or about 1:2 to about 2:1. In some examples, the at least one polyphenol and the at least one hydroxycinnamic acid may be present at a mass ratio ranging from 1:20 to about 20:1, or about 1:10 to about 10:1, about 1:5 to about 5:1, or about 1:2 to about 2:1. Any of the foregoing ranges may be utilized when the at least one polyphenol, the at least one hydroxychromane, and the at least one hydroxycinnamic acid are present in combination with one another. That is, the ratios and ranges may be independently varied when all three components are present.

The amounts of the at least one polyphenol, the at least one hydroxychromane, and the at least one hydroxycinnamic acid may be selected such that a concentration for each component is at or above a minimum concentration at which a synergistic increase in radical inhibition or radical quenching is realized in the cosmetic compositions described herein. The concentrations and relative ratios of these components may be within any of the ranges described above when achieving a synergistic interaction. If the concentrations of any one or more of the at least one polyphenol, the at least one hydroxychromane, and the at least one hydroxycinnamic acid are too low, the synergistic interaction may not occur or may not occur effectively. Conversely, if the concentrations of any one or more of the at least one polyphenol, the at least one hydroxychromane, and the at least one hydroxycinnamic acid are too high, the synergistic interaction may not be observable due to excessive signal, though the synergistic interaction may still be present. Thus, for in vitro analysis methods such as electron paramagnetic resonance (EPR) or RANDOX testing, low concentration ranges may be utilized due to the highly sensitive nature of these analyses, whereas higher concentration ranges may be more applicable for producing a desired effect during use.

Advantageously, the cosmetic compositions of the present disclosure may retain their radical-mitigating ability over extended periods of time. In non-limiting examples, the cosmetic compositions may quench 2,2-diphenyl-1-picrylhydrazyl radical (DPPH radical) more effectively after 3 months of aging than when freshly prepared. The DPPH efficacy may be about 15% or above after 3 months of aging, for example.

Isothermal titration calorimetry may also be utilized to establish minimum effective concentrations for the at least one polyphenol, the at least one hydroxychromane, and the at least one hydroxycinnamic acid in the cosmetic compositions described herein, particularly the minimum effective concentrations at which these components interact synergistically to mitigate radical formation or to promote quenching of previously formed radicals. Isothermal titration calorimetry measures ΔG, ΔH, and TΔS values, wherein ΔG is the change in free energy, ΔH is the change in enthalpy, and TΔS is the product of temperature (T) and the change in entropy (ΔS). The quantities are related by the expression ΔG=ΔH−TΔS. The dissociation constant (K_{d}) may also be calculated from isothermal titration calorimetry data by the expression ΔG=−RTInK_{d}, wherein R is the ideal gas constant (1.987×10⁻³ kcal/mol·K) and T is the temperature in Kelvin.

Negative values for ΔG are associated with favorable energetics and spontaneity of interactions or reactions. A negative value for ΔH and a positive value for TΔS contribute to a negative value for ΔG. Without being bound by theory or mechanism, negative values for ΔH are believed to arise from favorable hydrogen bonding interactions, and positive values for TΔS are believed to result from favorable hydrophobic interactions. If the concentration of one or more components is too low, the magnitude of the TΔS term may decrease (smaller positive value), thereby requiring the hydrogen bonding interaction associated with ΔH to be sufficiently large to overcome the hydrophobic interactions. Similarly, if the concentration of one or more components is too high, the ΔH may become positive, thereby requiring the hydrophobic interactions associated with TΔS sufficient to become sufficiently large overcome the poor hydrogen bonding interactions.

When ΔH and TΔS are negative and positive, respectively, both of these thermodynamic terms contribute favorably to a negative ΔG value, thereby ensuring reaction spontaneity. As such, isothermal titration calorimetry may be utilized to identify concentrations for the at least one polyphenol, the at least one hydroxychromane, and the at least one hydroxycinnamic acid at which ΔH and TΔS are negative and positive, respectively, and a spontaneous interaction between the components may occur. Thus, in some examples, the cosmetic compositions described herein may have concentrations of the at least one polyphenol, the at least one hydroxychromane, and the at least one hydroxycinnamic acid that are effective to produce a negative ΔH and a positive TΔS, as determined by isothermal titration calorimetry.

The cosmetic compositions of the present disclosure may be formulated in any liquid, semi-solid, or solid form, wherein suitable liquid forms may be an emulsion, serum, solution, or suspension, such as an aqueous emulsion or solution. Suitable liquid forms may comprise about 5 wt % to about 99.9 wt % water, based on total mass of the cosmetic compositions.

It is to be appreciated that additional ingredients that do not interfere with ability of the at least one polyphenol, the at least one hydroxychromane, and the at least one hydroxycinnamic acid to inhibit radical formation or to promote quenching of radicals. More particularly, any additional ingredients may be selected such that they do not compromise the ability of the at least one polyphenol, the at least one hydroxychromane, and the at least one hydroxycinnamic acid to interact synergistically with one another when accomplishing the foregoing. Non-limiting examples of such additional ingredients may include, for example, water, preservatives, other antioxidants, pH adjusters, solvents, oils, humectants, surfactants, solubilizers, conditioning agents, buffers, vitamins, emulsifiers, thickeners, chelating agents, preservatives, peptides, fragrances, minerals, colorants, waxes, clays, elastomers (including elastomer gels), particulates (such as mica and/or silica), conditioning agents, and inert ingredients such as silicones. Examples of such additional ingredients will be familiar to persons having ordinary skill in the art of formulating cosmetic compositions.

The cosmetic compositions of the present disclosure may be applied topically to a bodily structure, such as the skin or hair, as part of a user's self-care routine or when exposure to conditions that may promote radical formation upon or within the bodily structure is expected to take place (e.g., when sun exposure is expected) or has already taken place. The cosmetic compositions may be applied upon a desired bodily location one or more times a day, such in the morning and/or in the evening, or more frequently if needed or desired. When applied to the skin, the cosmetic compositions may or may not visibly alter the appearance of the skin.

Embodiments disclosed herein include the following:

Embodiment 1. A cosmetic composition comprising:
at least one polyphenol;
at least one hydroxychromane bearing at least one methyl group; and
at least one hydroxycinnamic acid.

Embodiment 2. The cosmetic composition of Embodiment 1, wherein the at least one polyphenol is present in green tea extract, resveratrol, or any combination thereof.

Embodiment 3. The cosmetic composition of Embodiment 1 or Embodiment 2, wherein the cosmetic composition comprises about 0.005 wt % to about 0.5 wt % of the at least one polyphenol, based on total mass of the cosmetic composition.

Embodiment 4. The cosmetic composition of Embodiment 1 or Embodiment 2, wherein the cosmetic composition comprises about 0.0009 wt % to about 0.0011 wt % of the at least one polyphenol, based on total mass of the cosmetic composition.

Embodiment 5. The cosmetic composition of any one of Embodiments 1-4, wherein the at least one hydroxychromane comprises copherol.

Embodiment 6. The cosmetic composition of Embodiment 5, wherein the at least one hydroxychromane comprises 90 wt % or greater of copherol, based on total mass of the at least one hydroxychromane.

Embodiment 7. The cosmetic composition of any one of Embodiments 1-6, wherein the cosmetic composition comprises about 0.005 wt % to about 0.5 wt % of the at least one hydroxychromane, based on total mass of the cosmetic composition.

Embodiment 8. The cosmetic composition of any one of Embodiments 1-6, wherein the cosmetic composition comprises about 0.0009 wt % to about 0.0011 wt % of the at least one hydroxychromane, based on total mass of the cosmetic composition.

Embodiment 9. The cosmetic composition of any one of Embodiments 1-8, wherein the at least one hydroxycinnamic acid comprises ferulic acid.

Embodiment 10. The cosmetic composition of any one of Embodiments 1-9, wherein the cosmetic composition comprises about 0.005 wt % to about 0.5 wt % of the at least one hydroxycinnamic acid, based on total mass of the cosmetic composition.

Embodiment 11. The cosmetic composition of any one of Embodiments 1-9, wherein the cosmetic composition comprises about 0.0009 wt % to about 0.0011 wt % of the at least one hydroxycinnamic acid, based on total mass of the cosmetic composition.

Embodiment 12. The cosmetic composition of any one of Embodiments 1-11, wherein the cosmetic composition quenches 2,2-diphenyl-1-picrylhydrazyl radical (DPPH radical) more effectively after 3 months of aging than when freshly prepared.

Embodiment 13. The cosmetic composition of any one of Embodiments 1-12, wherein a concentration of the at least one polyphenol, a concentration of the at least one hydroxychromane, and a concentration of the at least one hydroxycinnamic acid are each selected such that the cosmetic composition has a negative ΔG when evaluated by isothermal titration calorimetry, and optionally, a negative ΔH and a positive TΔS when evaluated by isothermal titration calorimetry.

Embodiment 14. The cosmetic composition of any one of Embodiments 1-13, wherein the at least one polyphenol, the at least one hydroxychromane, and the at least one hydroxycinnamic acid are present in the cosmetic composition at or above minimum concentrations effective to produce a synergistic increase in radical quenching or radical inhibition relative to the at least one polyphenol, the at least one hydroxychromane, and the at least one hydroxycinnamic acid alone at the same concentration.

Embodiment 15. A cosmetic composition comprising:
about 0.0005 wt % or above of at least one polyphenol;
about 0.0005 wt % or above of at least one hydroxychromane bearing at least one methyl group; and
about 0.0005 wt % or above of at least one hydroxycinnamic acid;
   wherein each wt % is based on total mass of the cosmetic composition.

Embodiment 16. The cosmetic composition of Embodiment 15, wherein the composition comprises about 0.005 wt % to about 0.5 wt % of the at least one polyphenol, about 0.005 wt % to about 0.5 wt % of the at least one hydroxychromane bearing at least one methyl group, and about 0.005 wt % to about 0.05 wt % of the at least one hydroxycinnamic acid.

Embodiment 17. The cosmetic composition of Embodiment 15 or Embodiment 16, wherein the at least one polyphenol is present in green tea extract, the at least one hydroxychromane comprises copherol, and the at least one hydroxycinnamic acid comprises ferulic acid.

Embodiment 18. The cosmetic composition of Embodiment 15 or Embodiment 16, wherein the at least one polyphenol comprises resveratrol, the at least one hydroxychromane comprises copherol, and the at least one hydroxycinnamic acid comprises ferulic acid.

Embodiment 19. The cosmetic composition of Embodiment 17 or Embodiment 18, wherein the at least one hydroxychromane comprises 90 wt % or greater of copherol, based on total mass of the at least one hydroxychromane.

Embodiment 20. The cosmetic composition of any one of Embodiments 15-19, wherein the cosmetic composition comprises about 0.0009 wt % to about 0.0011 wt % of the at least one polyphenol, about 0.0009 wt % to about 0.0011 wt % of the at least one hydroxychromane, and about 0.0009 wt % to about 0.0011 wt % of the at least one hydroxycinnamic acid, each based on total mass of the cosmetic composition.

Embodiment 21. The cosmetic composition of any one of Embodiments 15-20, wherein a concentration of the at least one polyphenol, a concentration of the at least one hydroxychromane, and a concentration of the at least one hydroxycinnamic acid are each selected such that the cosmetic composition has a negative ΔG when evaluated by isothermal titration calorimetry, and optionally, a negative ΔH and a positive TΔS when evaluated by isothermal titration calorimetry.

Embodiment 22. A method comprising:
applying the cosmetic composition of any one of Embodiments 1-21 to a bodily structure; and
inhibiting radical formation upon and/or within the bodily structure, and/or quenching radicals upon and/or within the bodily structure.

To facilitate a better understanding of the present disclosure, the following examples of preferred or representative embodiments are given. In no way should the following examples be read to limit, or to define, the scope of the invention.

### EXAMPLES

Example 1: Radical Inhibition (Randox Testing). Randox testing measures the percent inhibition of radical formation from the molecule ABTS (2,2′-azino-di-[3-ethylbenzthiazoline sulfonate]). Testing was performed using a Randox Laboratories testing kit under standard testing conditions. In brief, ABTS and various samples (see below) were incubated with a peroxidase (metmyoglobin) and H₂O₂ to produce the radical cation of ABTS. The radical cation has a blue-green color and is assayed spectrophotometrically at 600 nm. Inhibition of radical formation was determined by the spectrophotometric response for each sample based upon the extent of suppression of the absorbance at 600 nm relative to a deionized water blank.

The following samples were tested at a 0.001 wt % concentration of each component in deionized water: BHT (butylated hydroxytoluene), copherol, green tea extract, ferulic acid, 1:1 green tea extract/copherol, 1:1 ferulic acid/copherol, 1:1 green tea extract:ferulic acid, and 1:1:1 green tea extract/copherol/ferulic acid. The copherol was obtained as a vegetable oil solution (>80 wt % α-tocopherol based on total mass).

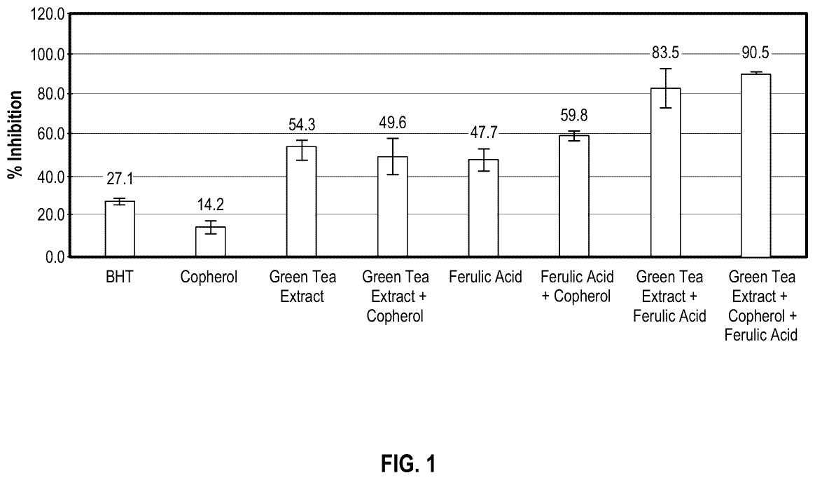

FIG. 1 is a bar graph showing the percent inhibition for formation of the ABTS radical cation for various samples. Each percent inhibition value was measured relative to a deionized water blank (0% inhibition). BHT, a known radical inhibitor, showed limited efficacy toward inhibition of ABTS radical cation formation. Copherol alone afforded even less radical inhibition than did BHT. Green tea extract alone and ferulic acid alone each afforded less than 55% inhibition. The binary combinations of 1:1 green tea extract:copherol and 1:1 copherol:ferulic acid afforded nearly the same degree of inhibition as did green tea extract alone and ferulic acid alone. Thus, copherol did not enhance the radical inhibition performance of green tea extract or ferulic acid.

The binary combination of 1:1 green tea extract:ferulic acid demonstrated a marked increase in inhibition that was approximately 30% higher (83.5%) than either green tea extract alone or ferulic acid alone. The ternary combination of 1:1:1 green tea extract:ferulic acid:copherol displayed a further increase in inhibition (90.5%). The further increase of the ternary combination is surprising, since copherol resulted in limited radical inhibition effects alone or in the 1:1 green tea extract:copherol sample. The inhibition increase produced by copherol in the ternary combination (7% increase) was about the same as that produced in the 1:1 ferulic acid:copherol sample (~12% increase). Collectively, the data suggests that green tea extract and ferulic acid interact synergistically with one another to inhibit radical formation. Further, there may be an additional synergistic interaction between ferulic acid and copherol to increase the radical inhibition performance still further in the ternary combination.

Example 2: Radical Inhibition (Randox Testing) in Formulated Samples. A water-in-oil base emulsion was obtained. The base emulsion base was modified with each of the materials specified in Example 1 (BHT, copherol, green tea extract, ferulic acid, 1:1 green tea extract/copherol, 1:1 ferulic acid/copherol, 1:1 green tea extract:ferulic acid, and 1:1:1 green tea extract/copherol/ferulic acid), with the loading of each component in the base emulsion again being held at 0.001 wt % relative to the total mass of the formulated sample. Radical inhibition for the formulated samples was evaluated by Randox testing as in Example 1.

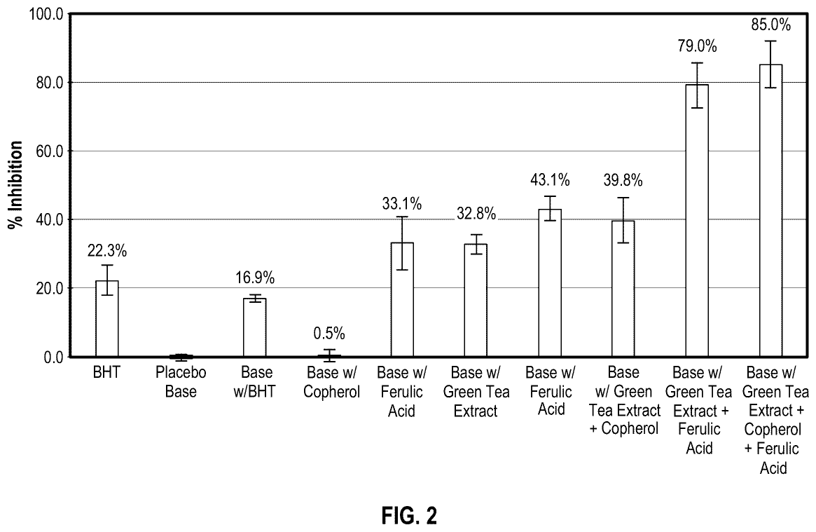

FIG. 2 is a bar graph showing the percent inhibition for formation of the ABTS radical cation for various formulated samples. As in Example 1, the binary combinations of 1:1 green tea extract/copherol and 1:1 ferulic acid/copherol afforded minimal enhancement of the radical inhibition. In contrast, and like in Example 1, the binary combination of 1:1 green tea extract:ferulic acid afforded significantly greater radical inhibition, and the ternary combination of 1:1:1 green tea extract:ferulic acid:copherol exhibited a still greater degree of radical inhibition.

Example 3: Radical Inhibition (Randox Testing) in Formulated Samples Having Different Concentrations of Active Components. Formulated samples having different concentrations of each component were also tested. Testing was conducted at a 10-fold lower concentration of components (0.0001 wt % for each component) and a 10-fold higher concentration of components (0.01 wt % for each component). At a concentration of 0.0001 wt % for each component, the percent inhibition was low, and the 1:1 green tea extract:ferulic acid and 1:1:1 green tea extract:ferulic acid:copherol samples failed to demonstrate a significant synergistic enhancement of the radical inhibition, as evaluated by Randox testing. At a concentration of 0.01 wt % for each component, all samples displayed high percent inhibition values, which were outside the calibration range of the test kit, thus making the synergistic interaction of the 1:1 green tea extract:ferulic acid and 1:1:1 green tea extract:ferulic acid:copherol samples unobservable by Randox testing. Percent inhibition values for the two sets of formulated samples are shown in Table 1.

TABLE 1

| | Percent Inhibition Values | | | | | | | |
|---|---|---|---|---|---|---|---|---|
| | Base | | | | | | | |
| | Emulsion | C | FA | GTE | T:FA | T:GTE | FA:GTE | GTE:T:FA |
| 0.0001 wt % | −13.1 | −7.2 | 33.3 | −10.5 | −4.2 | −12.5 | 38.9 | −6.7 |
| 0.001 wt % | −0.6 | 0.5 | 33.1 | 32.8 | 43.1 | 39.8 | 79 | 85 |
| 0.01 wt % | 24.1 | 20.4 | 128.7 | 144.8 | 116.1 | 154.0 | 122.0 | 133.4 |

C = copherol
FA = ferulic acid
GTE = green tea extract

Additional evaluation of the concentration-dependence of the various components is discussed further in Example 5 below.

Example 4: Radical Quenching (Spin Counts) in Formulated Samples. Formulated samples similar to those tested in Example 2 were used to study quenching of DPPH radical (2,2-diphenyl-1-picrylhydrazyl radical). Radical quenching was evaluated by electron spin resonance (ESR) spectroscopy to determine spin counts. Measurements were made using a Bruker MAGNETTECH ESR5000 instrument. Scans were conducted at room temperature every 20 seconds. The DPPH concentration was held at 1 mM in all samples. Spin count data was collected for both freshly prepared samples and samples aged following 3 months of storage at room temperature.

Tables 2A and 2B summarize the spin count data for various samples (freshly prepared, Table 2A; and aged for three months, Table 2B).

TABLE 2A

| | | Spin Counts as |
|---|---|---|
| | | Percentage of |
| Samples | | DPPH Spin Counts |
| (Freshly Prepared) | Spin Counts | Alone (%) |
| DPPH | 9.32 × 10¹⁵ | 100.00% |
| Placebo (Base Emulsion) | 9.08 × 10¹⁵ | 97.42% |
| BHT | 8.80 × 10¹⁵ | 94.4% |
| Copherol Alone | 7.57 × 10¹⁵ | 81.2% |
| Ferulic Acid Alone | 8.46 × 10¹⁵ | 90.8% |
| Green Tea Extract Alone | 7.77 × 10¹⁵ | 83.4% |
| 1:1 Ferulic Acid:Copherol | 7.85 × 10¹⁵ | 84.2% |
| 1:1 Green Tea Extract:Copherol | 7.17 × 10¹⁵ | 76.9% |
| 1:1 Green Tea Extract:Ferulic | 7.02 × 10¹⁵ | 75.3% |
| Acid | | |
| 1:1:1 Green Tea | 6.84 × 10¹⁵ | 73.4% |
| Extract:Copherol:Ferulic Acid | | |

TABLE 2B

| | | Spin Counts as |
|---|---|---|
| | | Percentage of |
| Samples | | DPPH Spin Counts |
| (Aged 3 Months) | Spin Counts | Alone (%) |
| DPPH | 1.05 × 10¹⁶ | 100.00% |
| Placebo (Base Emulsion) | 1.04 × 10¹⁶ | 99.1% |
| BHT | 9.83 × 10¹⁵ | 93.6% |
| Copherol Alone | 9.61 × 10¹⁵ | 91.5% |
| Ferulic Acid Alone | 8.86 × 10¹⁵ | 84.4% |
| Green Tea Extract Alone | 8.10 × 10¹⁵ | 77.1% |
| 1:1 Ferulic Acid:Copherol | 8.14 × 10¹⁵ | 77.5% |
| 1:1 Green Tea Extract:Copherol | 7.59 × 10¹⁵ | 72.3% |
| 1:1 Green Tea Extract:Ferulic Acid | 8.09 × 10¹⁵ | 77.1% |
| 1:1:1 Green Tea | 7.15 × 10¹⁵ | 68.1% |
| Extract:Copherol:Ferulic Acid | | |

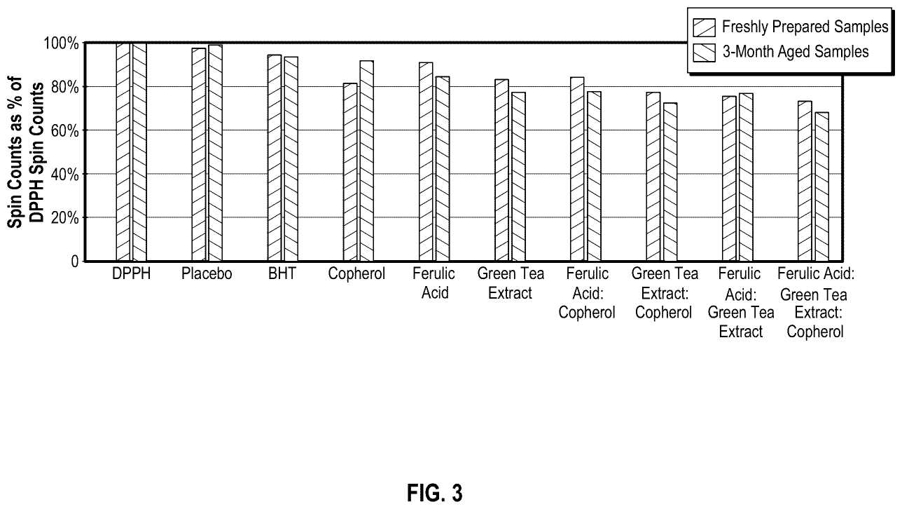

FIG. 3 is a bar graph of spin counts as a percentage of DPPH spin counts for freshly prepared samples and samples following three months of aging.

As in Example 1, the ternary combination of 1:1:1 green tea extract, copherol, and ferulic acid afforded the lowest spin counts relative to DPPH, followed closely by the binary combination of 1:1 green tea extract:ferulic acid. Surprisingly, the radical quenching effect of the ternary combination increased after 3 months of aging (lower spin counts). In contrast, the binary combination of 1:1 ferulic acid/green tea extract displayed increased spin counts relative to DPPH after 3 months of aging, indicating less effective radical quenching.

Example 5: Isothermal Titration calorimetry in Formulated Samples. Selected formulated samples having compositions similar to those tested in Example 2 were tested by isothermal calibration calorimetry (0.001 wt % of each component). Only freshly prepared binary and ternary combinations were tested by isothermal titration calorimetry.

Isothermal titration calorimetry measures ΔG, ΔH, and TΔS values, wherein ΔG is the change in free energy, ΔH is the change in enthalpy, and TΔS is the product of temperature (T) and the change in entropy (ΔS). The quantities are related by the expression ΔG=ΔH−TΔS. The dissociation constant (K_{d}) may also be calculated from isothermal titration calorimetry data by the expression ΔG=−RTInK_{d}, wherein R is the ideal gas constant (1.987×10−3 kcal/mol·K) and T is the temperature in Kelvin.

Negative values for ΔG are associated with favorable energetics and spontaneity of chemical interactions or reactions. A negative value for ΔH and a positive value for the product TΔS each contribute favorably to a negative value for ΔG, thereby ensuring spontaneity but not determinative of kinetic rate. Without being bound by theory or mechanism, negative values for ΔH and positive values for TΔS are believed to arise from a favorable balance of hydrogen bonding and hydrophobic interactions. ΔH is believed to relate primarily to hydrogen bonding interactions, and TΔS is believed to relate primarily to hydrophobic interactions. When ΔH and TΔS are not negative and positive, respectively (i.e., favorably signed), the magnitude of one favorably signed quantity must be relatively large to overcome the disfavorably signed quantity in order to afford a negative value of ΔG. For example, in the event ΔH is positive, TΔS must be a relatively large positive value to offset the effect of the positive ΔH in order to afford a negative value of ΔG. Similarly, in the event TΔS is negative, ΔH must be a relatively large negative value in order to offset the negative value of TΔS. Remaining unbound by theory, when ΔH and TΔS are not each favorably signed to contribute to a negative value of ΔG, binding may be dominated by hydrogen bonding or hydrophobic interactions in deference to the other. When ΔH and TΔS are favorably signed, in contrast, a better balance of hydrogen bonding and hydrophobic interactions may be realized.

Table 3 below shows isothermal titration calorimetry data for various freshly prepared formulated samples.

TABLE 3

| | | ΔG | ΔH | TΔS |
|---|---|---|---|---|
| | K_{d} (M) | (kcal/mol) | (kcal/mol) | (kcal/mol) |
| 1:1 Green Tea | 3.2 × 10⁻⁶ | −7.5 | +2.9 | +10.4 |
| Extract/Ferulic Acid | | | | |
| 1:1 Green Tea | 1.4 × 10⁻⁶ | −8.0 | +2.8 | +10.8 |
| Extract/Copherol | | | | |
| 1:1 Copherol/Ferulic | 9.2 × 10⁻⁸ | −9.6 | −16.1 | −6.5 |
| Acid | | | | |
| 1:1:1 Green Tea | 1.1 × 10⁻¹² | −16.3 | −1.2 | +15.1 |
| Extract/Copherol/ | | | | |
| Ferulic Acid | | | | |

As shown in Table 3, the ternary combination of 1:1:1 green tea extract/copherol/ferulic acid exhibited the most favorable thermodynamic quantities, as ΔH and TΔH were negative and positive, respectively. This sample exhibited a much smaller K_{d} value than did any of the binary combinations. The small K_{d} value is believed to be indicative of a high level of interactions between the three components of the ternary combination, which may explain the synergistic effects displayed in the Randox and spin count testing discussed above. In contrast to the ternary combination, the each of the binary combinations had one term disfavorably signed and resulting in a less negative ΔG value and correspondingly larger K_{d} value.

Example 6: Isothermal Titration calorimetry in Formulated Samples Having Different Concentrations of Components. Isothermal titration calorimetry of the ternary combination of 1:1:1 green tea extract/copherol/ferulic acid was also tested at different concentrations of each component. The concentrations of green tea extract, copherol, and ferulic acid parallel those of the concentration-dependent Randox testing discussed in Example 3 above, except formulated samples were analyzed, as in Example 5.

Table 4 below shows isothermal titration calorimetry data for various freshly prepared 1:1:1 green tea extract/copherol/ferulic acid formulated samples at various concentrations of components therein (0.0001 wt % and 0.01 wt %). The 0.001 wt % data from Example 5 is also shown in Table 4 for comparison.

TABLE 4

| | | ΔG | ΔH | TΔS |
|---|---|---|---|---|
| | K_{d} (M) | (kcal/mol) | (kcal/mol) | (kcal/mol) |
| 1:1:1 Green Tea | 2.7 × 10⁻⁵ | −6.3 | +80.0 | +86.3 |
| Extract/Copherol/ | | | | |
| Ferulic Acid | | | | |
| (0.01 wt %) | | | | |
| 1:1:1 Green Tea | 1.1 × 10⁻¹² | −16.3 | −1.2 | +15.1 |
| Extract/Copherol/ | | | | |
| Ferulic Acid | | | | |
| (0.001 wt %) | | | | |
| 1:1:1 Green Tea | 3.0 × 10⁻⁵ | −6.2 | −1.4 | +4.8 |
| Extract/Copherol/ | | | | |
| Ferulic Acid | | | | |
| (0.0001 wt %) | | | | |

As shown in Table 4, the thermodynamics of the ternary combination became less favorable as the concentration was varied. When the concentration was decreased from 0.001 wt % to 0.0001 wt %, ΔH decreased slightly but the TΔS became significantly less positive, thereby leading to a smaller negative value of ΔG and a correspondingly larger K_{d} value. In contrast, when the concentration was increased to 0.01 wt %, the ΔH term turned sharply positive. Although the change in sign of the ΔH term was offset by a corresponding increase in TΔS, a smaller negative value of ΔG again resulted. At the intermediate concentration value of 0.001 wt % for each component, hydrogen bonding and hydrophobic interactions were more favorably balanced, thereby leading to a larger negative ΔG value and a correspondingly smaller value of K_{d}. Again, it is to be appreciated that the thermodynamic results do not take into account the kinetic rate at which an interaction takes place.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element, or group of elements is preceded with the transitional phrase “comprising,” it is understood that we also contemplate the same composition or group of elements with transitional phrases “consisting essentially of,” “consisting of,” “selected from the group consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is the following:

1. A cosmetic composition comprising:
at least one polyphenol;
at least one hydroxychromane bearing at least one methyl group; and
at least one hydroxycinnamic acid.

2. The cosmetic composition of claim 1, wherein the at least one polyphenol is present in green tea extract, resveratrol, or any combination thereof.

3. The cosmetic composition of claim 2, wherein the cosmetic composition comprises about 0.005 wt % to about 0.5 wt % of the at least one polyphenol, based on total mass of the cosmetic composition.

4. The cosmetic composition of claim 2, wherein the cosmetic composition comprises about 0.0009 wt % to about 0.0011 wt % of the at least one polyphenol, based on total mass of the cosmetic composition.

5. The cosmetic composition of claim 1, wherein the at least one hydroxychromane comprises copherol.

6. The cosmetic composition of claim 5, wherein the cosmetic composition comprises about 0.005 wt % to about 0.5 wt % of the at least one hydroxychromane, based on total mass of the cosmetic composition.

7. The cosmetic composition of claim 5, wherein the cosmetic composition comprises about 0.0009 wt % to about 0.0011 wt % of the at least one hydroxychromane, based on total mass of the cosmetic composition.

8. The cosmetic composition of claim 1, wherein the at least one hydroxycinnamic acid comprises ferulic acid.

9. The cosmetic composition of claim 8, wherein the cosmetic composition comprises about 0.005 wt % to about 0.5 wt % of the at least one hydroxycinnamic acid, based on total mass of the cosmetic composition.

10. The cosmetic composition of claim 8, wherein the cosmetic composition comprises about 0.0009 wt % to about 0.0011 wt % of the at least one hydroxycinnamic acid, based on total mass of the cosmetic composition.

11. The cosmetic composition of claim 1, wherein the cosmetic composition quenches 2,2-diphenyl-1-picrylhydrazyl radical (DPPH radical) more effectively after 3 months of aging than when freshly prepared.

12. The cosmetic composition of claim 1, wherein a concentration of the at least one polyphenol, a concentration of the at least one hydroxychromane, and a concentration of the at least one hydroxycinnamic acid are each selected such that the cosmetic composition has a negative ΔG when evaluated by isothermal titration calorimetry, and optionally, a negative ΔH and a positive TΔS when evaluated by isothermal titration calorimetry.

13. The cosmetic composition of claim 1, wherein the at least one polyphenol, the at least one hydroxychromane, and the at least one hydroxycinnamic acid are present in the cosmetic composition at or above a minimum concentration effective to produce a synergistic increase in radical quenching or radical inhibition relative to the at least one polyphenol, the at least one hydroxychromane, and the at least one hydroxycinnamic acid alone at the same concentration.

14. A cosmetic composition comprising:
about 0.0005 wt % or above of at least one polyphenol;
about 0.0005 wt % or above of at least one hydroxychromane bearing at least one methyl group; and
about 0.0005 wt % or above of at least one hydroxycinnamic acid;
wherein each wt % is based on total mass of the cosmetic composition.

15. The cosmetic composition of claim 14, wherein the composition comprises about 0.005 wt % to about 0.5 wt % of the at least one polyphenol, about 0.5 wt % to about 0.05 wt % of the at least one hydroxychromane bearing at least one methyl group, and about 0.005 wt % to about 0.5 wt % of the at least one hydroxycinnamic acid.

16. The cosmetic composition of claim 14, wherein the at least one polyphenol is present in green tea extract, the at least one hydroxychromane comprises copherol, and the at least one hydroxycinnamic acid comprises ferulic acid.

17. The cosmetic composition of claim 14, wherein the at least one polyphenol comprises resveratrol, the at least one hydroxychromane comprises copherol, and the at least one hydroxycinnamic acid comprises ferulic acid.

18. The cosmetic composition of claim 14, wherein the cosmetic composition comprises about 0.0009 wt % to about 0.0011 wt % of the at least one polyphenol, about 0.0009 wt % to about 0.0011 wt % of the at least one hydroxychromane, and about 0.0009 wt % to about 0.0011 wt % of the at least one hydroxycinnamic acid, each based on total mass of the cosmetic composition.

19. The cosmetic composition of claim 14, wherein a concentration of the at least one polyphenol, a concentration of the at least one hydroxychromane, and a concentration of the at least one hydroxycinnamic acid are each selected such that the cosmetic composition has a negative ΔG when evaluated by isothermal titration calorimetry, and optionally, a negative ΔH and a positive TΔS when evaluated by isothermal titration calorimetry.

20. A method comprising:
applying the cosmetic composition of claim 16 to a bodily structure; and
inhibiting radical formation upon and/or within the bodily structure, and/or quenching radicals upon and/or within the bodily structure.
